(19) United States
(12) Patent Application Publication
(10) Pub. No.: US 2025/0110094 A1
(43) Pub. Date: Apr. 3, 2025

(54) MEASURING SYSTEM FOR A MONITORING SYSTEM

(71) Applicant: Dräger Safety AG & Co. KGaA, Lübeck (DE)

(72) Inventors: Tilman von BLUMENTHAL, Lübeck (DE); Hans-Ullrich HANSMANN, Lübeck (DE); Henning GERDER, Lübeck (DE); Robert JAHNS, Lübeck (DE); Christoph OSTERLOH, Lübeck (DE)

(21) Appl. No.: 18/895,598

(22) Filed: Sep. 25, 2024

(30) Foreign Application Priority Data
Sep. 28, 2023 (DE) 10 2023 126 404.4

(51) Int. Cl. G01N 33/00

(52) U.S. Cl. CPC G01N 33/0036

(57) ABSTRACT

A measuring system (60, 69, 68) determines gas concentrations as part of a monitoring system for monitoring the breathing gas supply of an aircraft pilot in an aircraft. The measuring system (60, 69, 68) is capable of determining an oxygen concentration (909) in the breathing gas mixture (10) of the aircraft pilot under variable operating temperatures (699) during flight operations of the aircraft.

### CROSS REFERENCE TO RELATED APPLICATIONS

This application claims the benefit of priority under 35 U.S.C. § 119 of German Application 10 2023 126 404.4, filed Sep. 28, 2023, the entire contents of which are incorporated herein by reference.

### TECHNICAL FIELD

The present invention relates to a paramagnetic measuring system for determining gas concentrations. In particular, the invention relates to a measuring system which is used as part of a monitoring system for monitoring the breathing gas supply with a sufficient concentration of oxygen of an aircraft pilot in an aircraft.

According to the invention, the measuring system is able to detect an oxygen concentration in the breathing gas mixture of the pilot during use of the aircraft in flight.

### BACKGROUND

Paramagnetic methods are often used to determine the oxygen concentration in gases, based on the fact that oxygen molecules are paramagnetic due to their permanent magnetic dipole moment, whereas most other gases have diamagnetic properties. It is generally known that the thermal conductivity of paramagnetic gases changes under the influence of magnetic fields. The cause of this behavior is apparently the fact that paramagnetic gases have a permanent magnetic moment which, however, does not normally appear (this is normally not observed) externally due to the thermal molecular motion of the gas molecules. However, a sufficiently strong external magnetic field ensures that the magnetic dipole moments of the individual molecules are aligned. On the one hand, this causes a change in susceptibility, which results in an increase in magnetic flux; on the other hand, a certain molecular arrangement is established in the gas, which limits the degrees of freedom and thus the possibilities of transferring thermal energy to neighboring molecules via collisions. This changes the thermal conductivity of the gas to a small extent. A basic principle for measuring oxygen in a measuring chamber using heat conduction changes in connection with paramagnetism is described in U.S. Pat. No. 6,430,987B1. US20230114548A1 describes a way of determining the presence of an anesthetic gas in the breathing gas mixture in addition to measuring oxygen.

A monitoring system for monitoring pilots and/or co-pilots is known from US20210405008A1. The monitoring system is preferably designed as part of the pilot's equipment as a self-sufficient and mobile unit worn on the body with an independent power supply.

Typical designs of paramagnetic measuring systems usually have a temperature control of the measuring chamber to a constant temperature well above the typical ambient temperatures in order to ensure stable conditions for the thermal conductivity measurements with and without the effect of the magnetic field. If a temperature level significantly above the usual operating temperature range is selected for measurement operation by means of temperature control to 55° C., for example, fluctuations in the ambient temperature have virtually no influence on the determination of the oxygen concentration. For use in a mobile monitoring system with an independent energy supply, this means that part of the mobile energy quantity—usually carried by a battery—must be kept available for temperature control. Operating a mobile system with temperature control therefore has a variety of consequences, particularly with regard to the size and weight of battery storage units or battery life, with an influence on the maximum possible operating time of such a monitoring system under these conditions.

### SUMMARY

Based on the state of the art, there is therefore a need for mobile measuring systems that are practicable in terms of size and weight, which are able to detect a concentration of oxygen with few restrictions in terms of duration of use.

The present invention is therefore based on the task of providing a measuring system with an optimization of the temperature control in order to overcome the above-mentioned disadvantages.

The problem is solved by a measuring system with features according to the invention.

Further features and details of the invention can be seen from this disclosure, including the claims, the description and the drawings.

A measuring system according to the invention has at least the following components for solving the tasks:
a gas delivery module,
a sample gas line (sample line/measurement gas line),
a measuring device
   with a measuring element in a measuring chamber,
   with a temperature sensor,
   with a magnet arrangement with an electromagnet and a coil,
a calculation and control unit with an assigned data memory, and
a circuit arrangement.

The gas delivery module is configured to draw in defined quantities or partial quantities of breathing gas mixture from a measuring location, in particular at the pilot's breathing mask and/or from the cabin or cockpit, by means of the sample gas line and to convey them to the monitoring system or into the measuring chamber. For this purpose, the gas delivery module is pneumatically and/or fluidically connected to the measuring location and the measuring chamber by means of the sample gas line.

The measuring element is configured as a planar semiconductor or silicon element (MEMS, semiconductor chip) with a membrane and has a heating structure on the membrane and a heat conduction measuring unit at a measuring point. The heat conduction measuring unit is configured as a thermocouple or as a thermopile. The heating structure can, for example, be configured as a resistive heating element. The calculation and control unit is configured to detect thermoelectric voltage signals of the measuring device provided by the measuring element.

The measuring device has a temperature sensor which is arranged in or on the measuring chamber or the measuring element in such a way as to detect a temperature value which indicates a temperature inside the measuring chamber or a temperature of a quantity of a breathing gas mixture inside the measuring chamber. The measuring device is configured with the temperature sensor to determine a temperature value which indicates a temperature inside the measuring chamber.

The measuring device, together with the circuit arrangement, is configured to heat the heating structure on the membrane of the measuring element. The measuring element is arranged in the measuring chamber in such a way that a quantity of the breathing gas mixture can be supplied to the measuring element as a gas sample. The measuring element is heated to a working temperature that is higher than the temperature of the quantity of breathing gas mixture in the measuring chamber. The measuring device is configured with the electromagnet, the coil and the circuit arrangement to generate a magnetic field acting on the measuring element. If a paramagnetic gas is present in the breathing gas mixture at the measuring element, the heat conduction changes under the influence of the magnetic field in proportion to the proportion of paramagnetic gas. If, for example, oxygen is present in the breathing gas mixture, the temperature rises, resulting in an increase in the thermoelectric voltage signals that can be detected at the heated measuring element. The calculation and control unit and the circuit arrangement can be used to monitor, control and regulate the temperature control of the measuring element:
to operate with a constant heating voltage,
to operate with a constant heating current, and
to operate with a constant heat output.

The circuit arrangement is configured for signal processing of the thermoelectric voltage signals with provision of measured values with an AC voltage signal component and with a DC voltage signal component to the calculation and control unit.

The signal processing uses suitable circuits, such as lock-in systems, high-pass/low-pass filter circuits, to split the thermoelectric voltage signals into an AC signal component and a DC signal component. Suitable variants for the operation of the measuring elements and the signal processing of the thermoelectric voltage signals can be found, for example, in US 2023 0114 548 A1 (US 2023 0114 548 A1 is incorporated herein by reference).

The circuit arrangement is configured to provide measured values of the measuring element with an alternating voltage (AC voltage) signal component U_{X˜} and with a direct voltage (DC voltage) signal component U_{X=} to the calculation and control unit.

The calculation and control unit is configured with the circuit arrangement to generate a modulated magnetic field acting on the measuring element by activating and deactivating the electromagnet with the coil arrangement. The calculation and control unit is configured with the circuit arrangement to split the thermoelectric voltage signals into alternating voltage signal components U_{X˜} and direct voltage signal components U_{X=}. The AC voltage signal components represent heat conduction components that are modulated and influenced by the magnetic field. Such an influence is given by the effect of paramagnetism in the heat conduction for the proportions of oxygen in the breathing gas mixture. The DC voltage signal components represent parts of the heat conduction in the breathing gas mixture that cannot be influenced by the magnetic field. The calculation and control unit is configured to determine the current oxygen concentration in the pilot's breathing gas mixture from the ratio of the modulated heat conduction components to the heat conduction components that are not modulated by the magnetic field. In the ideal case, the ratio of the modulated heat conduction components to the heat conduction components that are not modulated by the magnetic field represents the current oxygen concentration in cases where a binary gas mixture is given, i.e. for example a gas mixture consisting of a relevant proportion of oxygen and a predominant proportion of nitrogen with negligible proportions of carbon dioxide, noble gases and with a low water content, for example dry ambient air, technical or medical compressed air. In reality, however, a breathing gas mixture is not a binary gas mixture, as the concentrations of water vapor and carbon dioxide in particular are significantly higher during exhalation than during inhalation. To take into account gas mixtures, such as breathing gas mixtures with more than two relevant gas components, characteristic curves are required for a correction of the ratio of the modulated heat conduction components to the heat conduction components that are not modulated by the magnetic field. In addition, other physical conditions such as the absolute ambient pressure can be added as parameters in the configuration of the characteristic curves.

The number and selection of the required characteristic curves is crucial, but also means increasing complexity in systemic or individual calibration and in measurement operation. Coefficients for linear or higher derivation of the influences are preferably calculated from the characteristic curves.

For concentrations of oxygen (O₂) in a breathing gas mixture, the following approach for setting up characteristic curves according to the following formula 1 is used as an example:

$${C}_{O 2} = {K}_{1} * {a}_{1} / {b}_{1} + {K}_{2} * {( {a}_{1} / {b}_{1} )}^{2} + {K}_{3} * {a}_{1} + {K}_{4} * {a}_{1}^{2} + {K}_{5} * {b}_{1} + {K}_{6} * {b}_{1}^{2} + {K}_{7} * {c}_{1} + … ,$$

where K_{X} represents the coefficients of the components present in the breathing gas and the variables a₁, b₁, c₁, . . . each represent measured variables of heat conduction, such as the heat conduction dependent on the magnetic field and the heat conduction of the gases in the gas mixture independent of the magnetic field. In addition, the variables a₁, b₁, c₁, . . . can also include information on vapor pressures, dew points of the gases in the gas mixture or their water content in the gas mixture. These coefficients are available to the measuring system for the subsequent measurement operation.

According to the invention, the calculation and control unit is configured to include a temperature effect of the thermal conductivity in the determination of the current oxygen concentration in the breathing gas mixture of the pilot. In the configuration of the measuring system, characteristic curves are recorded which reflect, for example, the influence of material properties of the measuring elements, effects and boundary conditions of inflow, flow and/or outflow of supplied quantities of breathing gas mixture into the measuring chamber, of material properties and constructive structures of the measuring chamber or by processes of semiconductor production of the measuring elements (MEMS), for example in the form of possible scattering in the membrane thickness of the measuring elements.

In order to equalize or compensate for these influences, the thermovoltage signals with AC signal components and DC signal components and/or the ratios of AC signal components to DC signal components for typical breathing gas mixtures with oxygen components in the breathing gas mixture can be measured as part of a functional test for different compositions of the breathing gas mixture with proportions of oxygen, carbon dioxide, nitrogen, moisture or water vapor and can be stored and saved in a data memory (RAM, ROM) as data sets, which then characterize this measuring system individually or systemically in the form of a characteristic curve, a set of characteristic curves or as a characteristic diagram (characteristic field).

When the measuring system is used to determine gas concentrations when supplying an aircraft pilot, the data sets from the data memory are then used to determine the current oxygen concentration in the breathing gas mixture of the aircraft pilot with the highest possible accuracy from the AC signal components and DC signal components. The characteristic curves or the characteristic diagram are usually recorded under normal conditions of pressure and temperature or under typical defined ambient conditions of pressure and temperature, for example according to the approach described in Formula 1 for a temperature-controlled measuring system with a temperature level inside the measuring chamber that is stable at operating temperature. A suitable temperature level is formed above 35° C., for example at 37°, 45° C. or 50° C. If, as intended by the task of the present invention, the amount of energy required for temperature control is to be reduced or even largely omitted, a characteristic curve or a characteristic diagram based exclusively on measured values and data recorded at a single temperature level cannot achieve the same accuracy in determining the concentration of oxygen as in operation with temperature control of the measuring chamber at a stable 50° C., particularly if temperature control is omitted with then variable temperature conditions during operation. It is therefore necessary to take into account and include the temperature effect of the thermal conductivity of different gas proportions in the breathing gas mixture even for temperatures deviating from a stable temperature control of the measuring chamber when evaluating the signal and determining a current oxygen concentration in the breathing gas mixture. Operation with fluctuations in the temperature level inside the measuring chamber should also be possible. In measurement operation, the temperature sensor plays a key role in continuously determining the current temperature level inside the measurement chamber or the temperature level of the breathing gas mixture—as well as fluctuations in the temperature level—and providing it to the calculation and control unit.

The temperature effect can be included in accordance with the invention in the manner described below. According to the invention, the thermoelectric voltage signals are recorded to form characteristic curves or characteristic diagrams of the measuring elements at at least two defined and different temperature levels.

This can be done, for example, as part of a test of the measuring system following assembly or configuration or as part of a subsequent adjustment or calibration. The thermoelectric voltage signals with AC voltage and DC voltage signal components and/or the ratio of AC voltage to DC voltage signal component as a quotient are determined for different and typical compositions of moisture, oxygen, nitrogen, carbon dioxide in the breathing gas mixture as a first situation in the breathing gas mixture at a first temperature level and stored as a characteristic curve, a set of characteristic curves or a characteristic diagram as a first data set in the data memory. The thermovoltage signals with AC and DC voltage signal components and/or the ratio of AC voltage to DC voltage signal component as a quotient are determined for different and typical compositions of moisture, oxygen, nitrogen, carbon dioxide in the breathing gas mixture as a second situation in the breathing gas mixture at at least one further second temperature level and stored as a characteristic curve, a set of characteristic curves or a characteristic diagram as a second data set in the data memory.

The calculation and control unit is configured on the basis of the AC and DC voltage signal components to carry out a determination of a current oxygen concentration in the breathing gas mixture
including the temperature value, which indicates the current temperature level inside the measuring chamber,
including the first data set and
including the second data set.

The calculation and control unit is thus configured to determine an output signal, which indicates an oxygen concentration in the breathing gas mixture, on the basis of the AC and DC voltage signal components, the current temperature level inside the measuring chamber, at the measuring element or the amount of breathing gas mixture in the measuring chamber, as well as the first and second data sets. For this purpose, the calculation and control unit can apply corresponding characteristic curves for each of the at least two temperature levels according to the approach described above in formula 1.

For at least two further temperature levels, the coefficients can then be formed accordingly as coefficients Rₓ, Sₓ and the measured variables a₁, b₁, c₁ of the heat conduction for the at least two further temperature levels can be formed as a₂, b₂, c₂, or a₃, b₃, c₃ and stored accordingly in the first or second data set and kept ready for the measurement operation. The first temperature level is taken into account as the basis for the first data set, for example, using the approach shown in Formula 2 below:

$${C}_{O 2} = {R}_{1} * {a}_{2} / {b}_{2} + {R}_{2} * {( {a}_{2} / {b}_{2} )}^{2} + {R}_{3} * {a}_{2} + {R}_{4} * {a}_{2}^{2} + {R}_{5} * {R}_{1} + {R}_{6} * {b}_{2}^{2} + {R}_{7} * {c}_{2} + … .$$

The additional or second temperature level is taken into account as the basis for the second data set, for example, using the approach shown in Formula 3 below:

$${C}_{O 2} = {S}_{1} * {a}_{3} / {b}_{3} + {S}_{2} * {( {a}_{3} / {b}_{3} )}^{2} + {S}_{3} * {a}_{3} + {S}_{4} * {a}_{{3}^{2}} + {S}_{5} * {S}_{1} + {S}_{6} * {b}_{3}^{2} + {S}_{7} * {c}_{3} + … .$$

The calculation and control unit is configured to provide the output signal that indicates the specific oxygen concentration.

The following is a summarized description of the inventive embodiment of the measuring system for determining gas concentrations when supplying an aircraft pilot with a breathing gas mixture. The measuring system has a gas delivery module, a sample gas line and a measuring device.

The measuring device has a measuring element in a measuring chamber.

The gas delivery module is configured to supply a quantity of a breathing gas mixture to the measuring element in the measuring chamber.

The measuring device has a calculation and control unit with an associated data memory and a circuit arrangement. The measuring device also has an arrangement with an electromagnet and a coil.

The measuring device is configured with the circuit arrangement for operating a heating structure on a membrane of the measuring element in order to transfer defined quantities of heat into the breathing gas mixture or to partial quantities of the breathing gas mixture in the measuring chamber.

The measuring device is configured with the temperature sensor to determine a temperature value which indicates a temperature inside the measuring chamber.

The measuring device is configured with the electromagnet, the coil and the circuit arrangement for generating a magnetic field acting on the measuring element.

The circuit arrangement is configured to provide measured values of the measuring element with an AC voltage signal component and with a DC voltage signal component to the calculation and control unit.

A first data set with first values and at least one second data set with further values are stored in the data memory. The first data set indicates a situation of gases or breathing gas mixtures at a first temperature level. The second data set indicates a situation of gases or breathing gas mixtures at a second temperature level. In particular embodiments, at least one further or third data set with further values for a third or further temperature levels can be stored in the data memory, which indicate a third or further situation in the breathing gas mixture at a further temperature level.

The calculation and control unit is configured to determine a current oxygen concentration in the breathing gas mixture on the basis of the alternating voltage signal components, direct voltage signal components including the temperature value, which indicates a current temperature level inside the measuring chamber, including the first data set and the second data set.

The calculation and control unit is configured to provide an output signal that indicates the current oxygen concentration in the breathing gas mixture.

The following embodiments show three exemplary variants of how the calculation and control unit can be configured to include the current temperature value and the data sets stored in the data memory in measurement operation in the determination of the current oxygen concentration in the breathing gas mixture.

To explain a first variant, let us assume as an example that the first data set has a characteristic diagram with a temperature level of 25° C. as the data basis and the second data set has a characteristic diagram with a temperature level of 40° C. as the data basis. In this first example, the temperature level prevailing in measurement operation is given as 35° C. In this first variant, one approach would be to use the second data set in measurement operation as long as the current temperature level of the breathing gas mixture is above 30° C. and then switch to the first data set as soon as the current temperature level of the breathing gas mixture is below 31° C. In a corresponding preferred embodiment, the calculation and control unit is configured to include the current temperature value, the first data set and the second data set or the third or even further data sets by means of a switchover between the data sets to determine the current oxygen concentration.

To explain a second variant according to a further preferred embodiment, it is assumed by way of example that the first data set has a characteristic diagram with a temperature level of 25° C. as the data basis and the second data set has a characteristic diagram with a temperature level of 40° C. as the data basis. In this second example, the temperature level prevailing in measurement operation is given as 32° C.

In this second variant, one approach would be to carry out an interpolation of the data from the first and second data sets during measurement operation and to generate an interpolated data set during operation that is representative of the current temperature level of 32° C. with regard to different and typical compositions of moisture, oxygen, nitrogen and carbon dioxide in the breathing gas mixture.

Preferably, linear interpolation can be used for a number of two data sets.

To explain a third variant according to a further preferred embodiment, it is assumed by way of example that the first data set has a characteristic diagram with a temperature level of 25° C. as the data basis and the second data set has a characteristic diagram with a temperature level of 40° C. as the data basis. In this second example, the temperature level prevailing in measurement operation is given as 20° C.

In this third variant, one approach would be to extrapolate the data from the first and second data sets to the current temperature level of 20° C. during measurement operation and to generate an extrapolated data set during operation that is representative of the current temperature level of 20° C. with regard to different and typical compositions of humidity, oxygen, nitrogen and carbon dioxide in the breathing gas mixture.

Preferably, linear interpolation can be used for a number of two data sets and form preferred embodiments.

In further preferred embodiments, the thermoelectric voltage signals, their AC and DC voltage signal components and/or their quotient for different and typical compositions of oxygen, nitrogen and carbon dioxide in the breathing gas mixture can be determined for further temperature levels and stored as characteristic curves, characteristic curve sets or characteristic diagrams as further data sets in the data memory. This makes it possible, for example, to switch between different characteristic curves more finely based on the temperature value at the current measurement time in measurement operation. Alternatively, it can also be advantageously achieved, for example, that interpolations and extrapolations can be carried out on the basis of the temperature value at the current measurement time in measurement operation using a large number of interpolation points on the characteristic curves with improved approximation to the real temperature effect.

In addition, non-linear interpolations or extrapolations can also be used for more than three or five interpolation points

In a preferred embodiment, the interpolations or extrapolations may be performed as a linear interpolations or extrapolations or a sectional linear interpolations or extrapolations by the calculation and control unit.

In a preferred embodiment, the interpolation or the extrapolation can be performed as a non-linear interpolation or non-linear extrapolation, in particular as a quadratic or as a cubic interpolation or extrapolation by the calculation and control unit.

In a further preferred embodiment, the calculation and control unit can be configured to work together with a measuring chamber heater to control the temperature of the measuring chamber to a temperature level with a predefined temperature difference above the ambient temperature, for example above the temperature in the cockpit of the aircraft. In this way, condensation in the measuring chamber can be prevented with a low expenditure of heating energy. To implement this further preferred embodiment in practice, it is necessary to provide the calculation and control unit with a temperature value at an interface, which indicates the ambient temperature and/or the cockpit temperature.

In a further preferred embodiment, the calculation and control unit can be configured to initiate temperature control of the measuring chamber to a temperature level significantly above a typical body temperature of the pilot and the current ambient temperature for a predefined period of time after a predefined time interval has elapsed. In this way, condensation in the measuring chamber can be reliably prevented during the specified time period with a low expenditure of heating energy. In addition, the influence of the temperature dependencies of the thermal conductivities of the gases oxygen, nitrogen and carbon dioxide involved in the breathing gas mixture, as well as the influence of humidity on the determination of the current oxygen concentration in the breathing gas mixture, can be reduced for the duration of the specified time period.

In this way, a current oxygen concentration value of the breathing gas mixture can be determined from time to time, which is characterized by the fact that it has improved accuracy compared to the oxygen concentration value determined by means of switching, interpolations or extrapolations on the basis of the first and second data set.

In a preferred embodiment, the calculation and control unit can be configured in cooperation with a measuring chamber heating system to perform temperature control of the measuring chamber to a selected temperature level for a predefined period of time. The calculation and control unit can be configured to select the temperature level selected for the specified period of time using the current temperature value and at least one of the data sets stored in the data memory. This makes it possible, in an advantageous and energy-saving manner, to carry out the temperature control of the measuring chamber with a slight difference to the current temperature level, in that the temperature control of the measuring chamber is carried out at a temperature level which was also present when the first data set or the second data set or further data sets were generated.

Here is an example: The current temperature in the measuring chamber is 33° C., the first data set comprises a characteristic curve, a set of characteristic curves or a characteristic diagram with a reference temperature of 25° C., the second data set comprises a characteristic curve, a set of characteristic curves or a characteristic diagram with a reference temperature of 40° C. If the temperature of the measuring chamber is now stable at 40° C., the second data set can be used directly, i.e. without the use of interpolations or extrapolations. The calculation and control unit can then determine a current oxygen concentration in the breathing gas mixture from the thermoelectric voltage signals with AC voltage signal component and DC voltage signal component, taking into account the temperature value—in this example then 40° C.—exclusively using the second data set. In this way, a current oxygen concentration value of the breathing gas mixture can be determined continuously or only from time to time in measurement operation, which is characterized by the fact that it has an improved accuracy compared to the oxygen concentration value determined by means of switching, interpolation or extrapolations on the basis of the first and second data set, whereby only a small amount of energy is required for the temporary temperature control.

In a further preferred embodiment, the calculation and control unit can be configured to include in the determination of the oxygen concentration a measured value of a pressure sensor, which indicates a pressure level in the measuring chamber, and/or information provided with regard to a pressure level in the breathing gas mixture. In a comparable procedure to the approaches for different temperature levels shown with formulae 1, 2, 3 as a basis for realizing characteristic curves, characteristic diagrams or sets of characteristic curves stored in data sets in the data memory, further approaches with formulae 4 and 5 can be specified in this preferred embodiment in order to additionally include at least two different pressure levels in the determination of the oxygen concentration as data sets with interpolations, extrapolations based on the data sets or switching between the data sets.

For two different pressure levels, for example, the coefficients can be formed accordingly as coefficients Pₓ, Qₓ and the measured variables a₁, b₁, c₁ of the heat conduction for the two pressure levels can be formed as a₂, b₂, c₂, or a₃, b₃, c₃ and stored accordingly in further data sets and kept ready for measurement operation. Consideration of a pressure level as the basis for a third data set is carried out, for example, using the approach according to the following formula 4:

$${C}_{O 2} = {P}_{1} * {a}_{2} / {b}_{2} + {P}_{2} * {( {a}_{2} / {b}_{2} )}^{2} + {P}_{3} * {a}_{2} + {P}_{4} * {a}_{2}^{2} + {P}_{5} * {P}_{1} + {P}_{6} * {b}_{2}^{2} + {P}_{7} * {c}_{2} + … .$$

A further pressure level is taken into account as the basis for a fifth data set, for example, using the approach shown in Formula 5 below:

$${C}_{O 2} = {Q}_{1} * {a}_{3} / {b}_{3} + {Q}_{2} * {( {a}_{3} / {b}_{3} )}^{2} + {Q}_{3} * {a}_{3} + {Q}_{4} * {a}_{3}^{2} + {Q}_{5} * {Q}_{1} + {Q}_{6} * {b}_{3}^{2} + {Q}_{7} * {c}_{3} + … .$$

In a further preferred embodiment, the calculation and control unit can be configured to include in the determination of the oxygen concentration a measured value of a humidity sensor, which indicates a humidity situation in the breathing gas mixture in the measuring chamber, and/or information provided with regard to a current humidity content in the breathing gas mixture.

The current moisture content in the breathing gas mixture can, for example, be included in the data sets based on formulas 1 to 5 or directly or indirectly in formulas 1 to 5, which have the measured variables of heat conduction a₁, a₂, a₃, b₁, b₂, b₃, c₁, c₂, c₃ as parameters or information on vapor pressures, dew points of the gases in the gas mixture or their water content in the gas mixture.

In a further preferred embodiment, a pressure sensor can be arranged in or on the measuring chamber.

In a further preferred embodiment, a humidity sensor can be arranged in or on the measuring chamber.

The invention is explained in more detail in the following description with partial reference to the figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view of a monitoring system showing some features of a measuring system for determining a concentration; and

FIG. 2 is a schematic view showing further details of the measuring system for determining a concentration.

### DESCRIPTION OF PREFERRED EMBODIMENTS

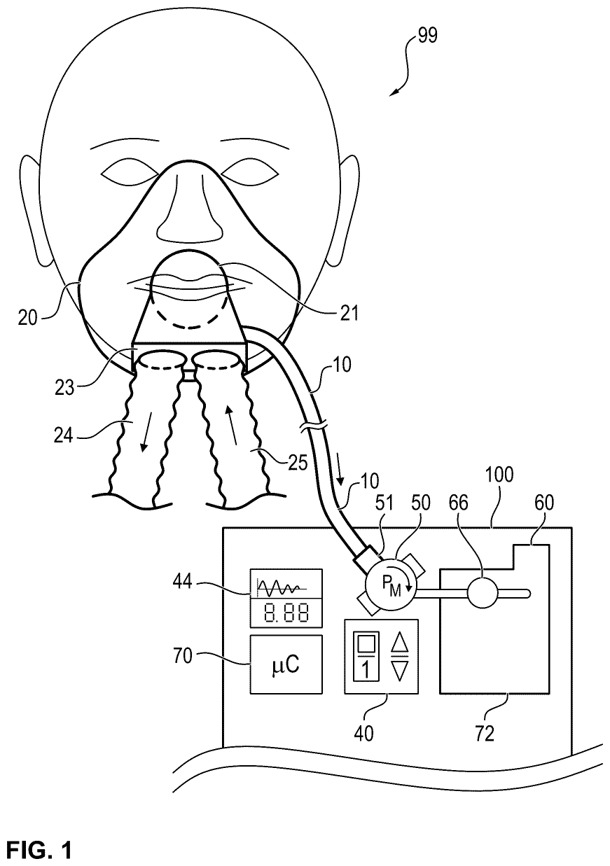

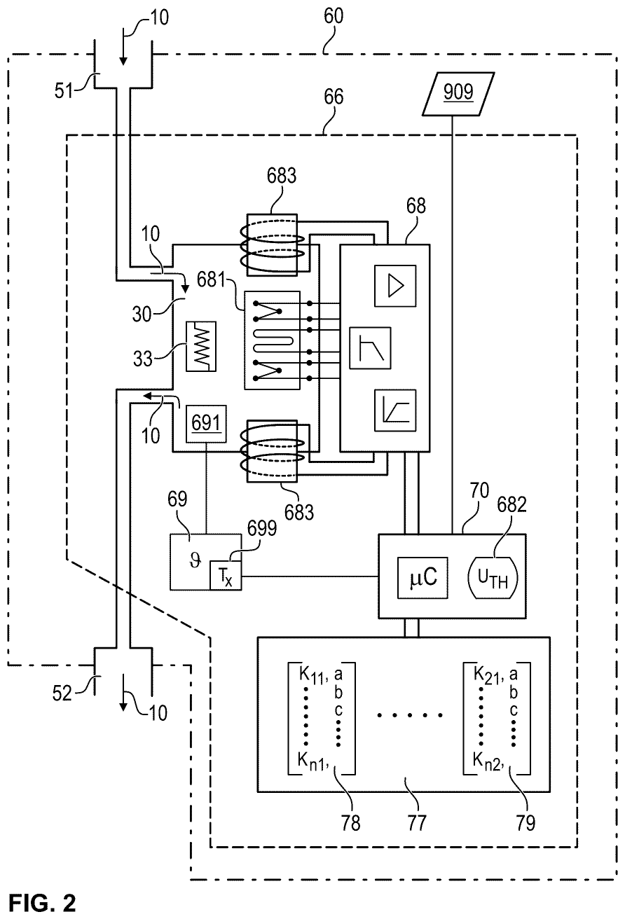

Referring to the drawings, identical elements in FIGS. 1 and 2 are designated with identical reference numerals in FIGS. 1, 2. FIG. 1 shows a monitoring system 100, showing some features of the measuring system for determining a concentration according to the invention.

The monitoring system 100 is connected to a person 99 with a sample gas line (measurement gas line) 10 by means of a breathing mask 20. The person 99 is an airplane pilot (aviators, pilots, co-pilots). The breathing mask 20 has a gas connection 21, a connection element 23 and hose lines 24, 25. The hose lines 24, 25 are used to remove and supply breathing gases to the person 99.

The monitoring system 100 has operating elements 40, display elements 44, a gas delivery module 50, a measuring system 60 with a measuring device 66.

The measuring device 66 has a sensor system, in particular a sensor system for gas analysis, but can also have other sensor systems, for example a sensor system that is configured to measure temperatures, pressures or humidity. The gas delivery module 50 is preferably configured as a pump P_{M}.

In addition, the monitoring system 100 has a control and evaluation unit 70. The operating elements 40, the display elements 44, the sensors and the gas delivery module 50 are connected to the control and evaluation unit 70 via signal and data lines or control lines, which are not shown in FIG. 1. These control lines or signal and data lines can, for example, be configured as a bus system (CAN) or network.

The control and evaluation unit 70 is configured and intended to control and/or actuate the gas delivery module 50 in such a way that breathing gases are delivered from the breathing mask 20 through the sample gas line 10 and a gas inlet 51 to the measuring system 60. Thus, a quantity or partial quantity of breathing gas 10 is then available to the measuring device 66 in order to analyze it metrologically and provide it to the control and evaluation unit 70 as measured values.

The control and evaluation unit 70 enables the measured values to be evaluated, processed and displayed on display elements 44.

FIG. 2 shows the measuring system 60 with the measuring device 66 and the control and evaluation unit 70 according to FIG. 1 in detail, as well as with an additional data memory 77, which is provided and suitable for storing a first data record 78 and at least one further data record 79 and making them available to the control and evaluation unit 70. Quantities of breathing gas mixture 10 can be conveyed by the gas delivery module 50 (FIG. 1) via a gas inlet 51 to and into a measuring chamber 30 and out again via a gas outlet 52.

The measuring system 60 has the measuring device 66 with a circuit arrangement 68, which is configured to determine a concentration of oxygen (O₂) in the breathing gas mixture 10 on the basis of paramagnetic effects of thermal conductivity.

The circuit arrangement 68 has a magnet arrangement 683 with an electromagnet and a coil as well as electronic components for operating the coil, magnetic field and for carrying out signal processing and signal filtering. The measuring device 66 has a measuring element 681 with heating elements, thermocouples or thermopiles, which are arranged on a membrane. By means of the interaction of the control and evaluation device 70, circuit arrangement 68 and a magnet arrangement 683, a cyclical effect of a magnetic field on the measuring element 681 is generated during operation. In addition to the elements schematically indicated in FIG. 2 for guiding a magnetic field, the magnet arrangement 683 has an electromagnet with coils in a coil arrangement, which—like the magnetic field itself—are only indicated in this FIG. 2 for reasons of clarity.

Thermoelectric voltage signals 682 are determined at the thermocouples or thermopiles with and without the influence of the magnetic field as signals with a DC voltage component U_{TH=} and with an AC voltage component U_{TH˜}.

The thermoelectric voltage signals determined at the thermocouples or thermopiles 682 U_{TH=}, U_{TH˜}, indicate a thermal conductivity of the breathing gas mixture 10 upon contact with the measuring element 681. The thermoelectric voltage signals 682 U_{TH=}, U_{TH˜} are provided to the control and evaluation unit 70. The measuring device 66 has a temperature measuring arrangement 69 with an associated temperature sensor 691 arranged in or on the measuring chamber 30 for determining a temperature level in the breathing gas mixture 10 as well as its changes in the breathing gas supply to the pilot 99 (FIG. 1). The control and evaluation unit 70 is configured to continuously detect a temperature level inside the measuring chamber 30 as a current temperature value Tₓ 699 from signals of the temperature sensor 691, which represents a temperature level in the breathing gas mixture 10.

An optional measuring chamber heater 33 is arranged on the measuring chamber 30, which is configured to cause at least temporary heating of the measuring chamber 30 in cooperation with the calculation and control unit 70 in order to reduce condensation of moisture in the measuring chamber 30, at least temporarily.

The first data set 78 contains a characteristic curve for various typical proportions of moisture and the gases nitrogen, oxygen and carbon dioxide in the breathing gas mixture 10 with reference to a first temperature level.

The second data set 79 contains a characteristic curve for typical proportions of moisture and the gases nitrogen, oxygen and carbon dioxide in the breathing gas mixture 10 with reference to a further temperature level.

The control and evaluation unit 70 is configured to evaluate the thermoelectric voltage signals 682 U_{TH=}, U_{TH} on the basis of the data of the first data set 78 and the data of the further data set 79, taking into account the current temperature level Tₓ 699, i.e. to form a ratio of the DC voltage component U_{TH=} and the AC voltage component U_{TH˜} in order to determine a current concentration value of oxygen (O₂) in the breathing gas mixture 10 and to provide it as an output signal 909, which indicates the current concentration value of oxygen in the breathing gas mixture 10, and to provide it to an output unit 44 (FIG. 1) or to an optional interface.

In optional embodiments, the measuring system 60 may have further measuring arrangements for temperature and/or pressure measurements and/or humidity measurements.

While specific embodiments of the invention have been shown and described in detail to illustrate the application of the principles of the invention, it will be understood that the invention may be embodied otherwise without departing from such principles.

### LIST OF REFERENCE SYMBOLS

10 Measuring line for breathing gas mixture
20 Breathing mask
21 Gas connection to breathing mask
23 Connecting element
24,25 Hose lines (hoses)
30 Measuring chamber
40 Input elements
44 Display elements (indicators)
50 Gas delivery module, pump P_{M}
51 Gas inlet
52 Gas outlet
60 Measuring system
66 Measuring device, sensors
68 Circuit arrangement
681 Measuring element with heating and thermocouple arrangement
682 Thermoelectric voltage signals U_{TH}
683 Magnet arrangement
69 Temperature measuring arrangement
691 Temperature sensor
699 Temperature value
70 Control and evaluation unit
77 Data memory
78, 79 Data set, data sets
99 Person, pilot, airplane pilot
100 Monitoring system
909 Output signal

What is claimed is:

1. A measuring system for determining gas concentrations when supplying an aircraft pilot with a breathing gas mixture in an aircraft, the measuring system comprising:
a gas delivery module;
a sample gas line;
a measuring device comprising a measuring element in a measuring chamber, a temperature sensor, a magnet arrangement with an electromagnet and a coil, a calculation and control unit with an associated data memory and a circuit arrangement,
wherein the gas delivery module is configured to supply a quantity of a breathing gas mixture to the measuring element in the measuring chamber,
wherein the measuring device with the circuit arrangement is configured to operate a heating structure on a membrane of the measuring element in order to transfer defined quantities of heat into the breathing gas mixture or to partial quantities of the breathing gas mixture in the measuring chamber,
wherein the measuring device with the temperature sensor is configured to determine a temperature value which indicates a temperature inside the measuring chamber,
wherein the measuring device with the electromagnet, the coil and the circuit arrangement is configured to generate a magnetic field acting on the measuring element,
wherein the circuit arrangement is configured to provide measured values of the measuring element, with an AC voltage signal component and with a DC voltage signal component, to the calculation and control unit,
wherein a first data set with first data and at least one second data set with further data are stored in the data memory,
wherein the first data set indicates a first situation in the breathing gas mixture at a first temperature level,
wherein the second data set indicates a second situation of gases or breathing gas mixture at a second temperature level,
wherein the calculation and control unit is configured to determine a current oxygen concentration in the breathing gas mixture based on the alternating voltage signal components and direct voltage signal components and including the temperature value, which indicates the current temperature level inside the measuring chamber, and including the first data set and including the second data set, and
wherein the calculation and control unit is configured to provide an output signal which indicates the determined current oxygen concentration in the breathing gas mixture.

2. A measuring system according to claim 1, wherein at least one further or third data set with further values for a third or further temperature levels is stored in the data memory, and wherein the at least one further or third data set indicates a third or further situation in the breathing gas mixture at the third or further temperature levels.

3. A measuring system according to claim 1, wherein the calculation and control unit is configured to include the current temperature value, the first data set and the second data set, by means of a switchover between the data sets, to determine the current oxygen concentration.

4. A measuring system according to claim 2, wherein the calculation and control unit is configured to include the current temperature value, the first data set, the second data set and/or the third or also further data sets, by means of a switchover between the data sets, to determine the current oxygen concentration.

5. A measuring system according to claim 1, wherein the calculation and control unit is configured to include the first data set and the second data set, by means of an interpolation based on the values of the data sets for the current temperature value, to determine the current oxygen concentration.

6. A measuring system according to claim 2, wherein the calculation and control unit is configured to include the first data set, the second data set and/or the third data set or also further data sets, by means of an interpolation based on the values of the data sets for the current temperature value, to determine the current oxygen concentration.

7. A measuring system according to claim 1, wherein the calculation and control unit is configured to include the first data set and the second data set, by means of an extrapolation based on the values of the data sets for the current temperature value, to determine the current oxygen concentration.

8. A measuring system according to claim 2, wherein the calculation and control unit is configured to include the first data set and the second data set or the third data set or also further data sets, by means of an extrapolation based on the values of the data sets for the current temperature value, to determine the current oxygen concentration.

9. A measuring system according to claim 5, wherein the calculation and control unit is configured to perform the interpolation as a linear interpolation or a sectional linear interpolation or to perform the interpolation as a non-linear interpolation, as a quadratic interpolation or as a cubic interpolation.

10. A measuring system according to claim 7, wherein the calculation and control unit is configured to perform the extrapolation as a linear extrapolation or a sectional linear extrapolation or to perform the extrapolation as a non-linear extrapolation, as a quadratic extrapolation or as a cubic extrapolation.

11. A measuring system according to claim 1, wherein the calculation and control unit is configured, in cooperation with a measuring chamber heater, to perform a temperature control of the measuring chamber to a temperature level with a predefined temperature difference above an ambient temperature or a temperature control of the measuring chamber to a temperature level above a body temperature of the aircraft pilot for a predefined period of time.

12. A measuring system according to claim 1, wherein the calculation and control unit is configured, in cooperation with a measuring chamber heater, to carry out temperature control of the measuring chamber to a selected temperature level for a predefined period of time, wherein the calculation and control unit is configured to select the temperature level selected for the predefined period of time on the basis of the current temperature value and on the basis of at least one of the data sets stored in the data memory.

13. A measuring system according to claim 1, wherein the calculation and control unit is configured to include in the determination of the oxygen concentration: a measured value of a pressure sensor, which indicates a pressure level in the measuring chamber; and/or information provided with regard to a pressure level in the breathing gas mixture, and/or a measured value of a humidity sensor, which indicates a humidity situation in the breathing gas mixture in the measuring chamber, and/or information provided with regard to a current humidity level in the breathing gas mixture, and/or a measured value of a humidity sensor, which indicates a humidity situation in the breathing gas mixture in the measuring chamber, and/or information provided with regard to a current humidity content in the breathing gas mixture.

14. A measuring system according to claim 13, wherein a pressure sensor is arranged in or on the measuring chamber, which pressure sensor indicates a pressure level in the measuring chamber and/or wherein a humidity sensor is arranged in or on the measuring chamber, which indicates a humidity level in the measuring chamber.
